(19) United States
(12) Patent Application Publication
(10) Pub. No.: US 2026/0014088 A1
(43) Pub. Date: Jan. 15, 2026

(54) LIPID NANOPARTICLES FOR PROTEIN DELIVERY

(71) Applicants: PANGEN BIOTECH INC., Gyeoggi-do (KR); MOOGENE MEDI CO., LTD., Gyeonggi-do (KR)

(72) Inventors: Jaeseung YOON, Gyeonggi-do (KR); Tae Jong YOON, Seoul (KR)

(21) Appl. No.: 18/994,353

(22) PCT Filed: Jul. 14, 2023
(86) PCT No.: PCT/KR2023/010139

§ 371 Date: Jan. 14, 2025

(30) Foreign Application Priority Data
Jul. 15, 2022 (KR) 10-2022-0087733

(51) Int. Cl. A61K 9/51; A61K 38/46

(52) U.S. Cl. CPC A61K 9/5123; A61K 9/5146; A61K 38/465

(57) ABSTRACT

The present invention relates to lipid nanoparticles for the delivery of proteins and, specifically, to lipid nanoparticles for the intracellular or in vivo delivery of various medicinal proteins such as therapeutic proteins or enzymes for enzyme replacement therapy (ERT). The present invention can be advantageously used for the treatment and prevention of various diseases, including lysosomal storage disorders, by stably and effectively delivering various proteins such as therapeutic proteins or ERT enzymes into cells through lipid nanoparticles.

### DESCRIPTION

### Technical Field

The present disclosure relates to a lipid nanoparticle for protein delivery, and more particularly, to a lipid nanoparticle for the intracellular or in vivo delivery of various medicinal proteins, such as therapeutic proteins or enzymes for enzyme replacement therapy (ERT).

### Background Art

Lipid nanoparticles (LNPs) have recently attracted much attention as a system for effectively delivering biomolecules such as mRNA in the body, and are composed of phospholipids, cholesterol, various ionic lipids, PEG-containing phospholipids, etc. to stably deliver substances into the body.

Meanwhile, lysosomal storage disorders (LSDs) are relatively rare inherited metabolic disorders caused by defects in lysosomal function. LSDs are typically caused by a deficiency of a single enzyme that participates in the breakdown of metabolites in lysosomes. The accumulation of products due to the lack of enzyme activity can affect various organ systems and lead to severe symptoms and early death. The majority of LSDs also have significant neurological deficits ranging from progressive neurodegeneration, severe cognitive impairment to epilepsy, behavioral disorders, and psychiatric disorders.

Representative examples of these lysosomal storage disorders include Fabry disease, Gaucher disease, and mucopolysaccharidosis, etc., and enzyme replacement therapy (ERT) has been established as a standard treatment. This enzyme replacement therapy serves to lower the concentration of harmful substances accumulated in cells by regularly intravenously administering enzyme proteins that are deficient in the body (Concolino et al., Italian Journal of Pediatrics, 2018).

However, in order to improve the therapeutic efficiency of such an enzyme replacement therapy, it is necessary to develop a delivery system for reducing side effects such as unwanted immune responses and for effective and stable intracellular delivery of enzyme proteins. This is to lower the concentration of the enzyme administered or to minimize anti-drug antibody reactions.

### DISCLOSURE

### Technical Problem

An object of the present disclosure is to provide a lipid nanoparticle for intracellular or in vivo delivery of a protein.

Another object of the present disclosure is to provide a lipid nanoparticle encapsulated with a protein.

Another object of the present disclosure is to provide a composition for intracellular or in vivo delivery of a protein comprising a lipid nanoparticle encapsulated with a protein.

Another object of the present disclosure is to provide a pharmaceutical composition for preventing or treating a lysosomal storage disorder, comprising a lipid nanoparticle encapsulated with a protein.

Another object of the present disclosure is to provide a method for preparing a lipid nanoparticle encapsulated with a protein.

Another object of the present disclosure is to provide a method for intracellular delivery of an ERT enzyme or a therapeutic protein using a lipid nanoparticle.

Another object of the present disclosure is to provide a method for treating a disease using a lipid nanoparticle encapsulated with an ERT enzyme or a therapeutic protein.

It will be appreciated that the technical problems to be solved in accordance with the technical ideas of the disclosure disclosed herein are not limited to those problems mentioned above, and other problems not mentioned will be apparent to those of ordinary skill in the art from the following description.

### Technical Solution

This will be specifically described as follows. Meanwhile, each of the descriptions and embodiments disclosed in the present application may also be applied to each other descriptions and embodiments. That is, all combinations of various elements disclosed in the present application fall within the scope of the present application. In addition, the scope of the present application is not to be considered limited by the specific description set forth below.

As an aspect to achieve the objectives of the present disclosure, the present disclosure provides a lipid nanoparticle for in vivo delivery of a protein. Specifically, the present disclosure provides a lipid nanoparticle for intracellular or in vivo delivery of a protein, comprising a lipid mixture including hydrogenated soybean phosphatidylcholine (HSPC); cholesterol; dioleoyl-3-trimethylammonium propane (DOTAP); and 1,2-distearoyl-sn-glycero-3-phosphoethanolamine-N-methoxy (polyethylene glycol) (DSPE-mPEG).

The term “lipid nanoparticle (LNP)” as used herein refers to an endoplasmic reticulum having an adjacent lipid bilayer, such as a spherical endoplasmic reticulum, and may be used for the delivery to a targeted location. These lipid nanoparticles may comprise liposomes, solid lipid nanoparticles (SLNs), nanostructured lipid carriers (NLCs), and monolayer membrane structures (micelles).

The lipid nanoparticle of the present disclosure may comprise one or more types of lipids. For example, it may include ionic lipids, PEG-lipids, phospholipids, or cholesterol, etc. Here, the ionic lipid may be a cationic lipid. The cationic lipid includes, for example, N-(2,3-dioleoyloxy)propyl)-N,N,N-trimethylammonium chloride (DOTAP); N-(2,3-dioleoyloxy)propyl)-N,N,N-trimethylammonium chloride (DOTMA); N,N-distearyl-N,N-dimethylammonium bromide (DDAB); N,N-dioleyl-N,N-dimethylammonium chloride (DODAC); N-(1,2-dimyristyloxyprop-3-yl)-N,N-dimethyl-N-hydroxyethylammonium bromide (DMRIE); N1,N3,N5-tris (3-(didodecylamino)propyl)benzene-1,3,5-tricarboxamide (TT3), lipofectamine; 1,2-dilinoleyloxy-N,N-dimethylaminopropane (DLinDMA), 1,2-dilinolenyloxy-N,N-dimethylaminopropane (DLenDMA), dioctadecyldimethylammonium (DODMA), distearyldimethylammonium (DSDMA), 3-(N-(N′,N′-dimethylaminoethane)-carbamoyl) cholesterol (DC-Chol), and 1,2-dioleolyl-3-dimethylammonium propane (DODAP), etc.

It may also be composed of other lipid components. For example, it may include other lipid molecules belonging to the phosphatidylcholine (PC) class (e.g., 1,2-distearoyl-sn-glycero-3-phosphocholine (DSPC), and 1,2-dioleoyl-sn-glycero-3-phosphoethanolamine (DOPE), sterols (e.g., cholesterol) and polyethylene glycol (PEG)-lipid conjugates (e.g., 1,2-distearoyl-sn-glycero-3-phosphoethanolamine-N-[polate(polyethylene glycol)-2000 (DSPE-PEG2000), 1,2-distearoyl-sn-glycero-3-phosphoethanolamine-N-methoxy(polyethylene glycol) (DSPE-mPEG), L-alpha-phosphatidylcholine (HSPC), dipalmitoyl phosphatidylcholine (DPPC), and 1,2-dimyristoyl-sn-glycero-3-phosphoethanolamine-N-[methoxy (polyethylene glycol)-2000 (C14-PEG2000)).

Specifically, the lipid nanoparticle of the present disclosure may comprise a lipid mixture including HSPC; cholesterol; DOTAP; and DSPE-mPEG.

The ratio of each of these lipids in the lipid nanoparticle is not particularly limited, but for example, the HSPC may be included in an amount of 8 to 15 mol % of the lipid mixture in the lipid nanoparticle, the cholesterol may be included in an amount of 30 to 50 mol % of the lipid mixture in the lipid nanoparticle, the DOTAP may be included in an amount of 40 to 60 mol % of the lipid mixture in the lipid nanoparticle, and DSPE-mPEG may be included in an amount of 0.5 to 5 mol % of the lipid mixture in the lipid nanoparticle. More preferably, the HSPC may be included in an amount of 9 mol % of the lipid mixture in the lipid nanoparticle, the cholesterol may be in included in an amount of 40 mol % of the lipid mixture in the lipid nanoparticle, the DOTAP may be included in an amount of 50 mol % of the lipid mixture in the lipid nanoparticle, and DSPE-mPEG may be included in an amount of 1 mol % of the lipid mixture in the lipid nanoparticle.

In addition, the lipid mixture may be comprised of HSPC:cholesterol:DOTAP:DSPE-mPEG in a molar ratio of 8 to 15:30 to 50:40 to 60:0.5 to 5, preferably, the lipid mixture may be comprised of HSPC:cholesterol:DOTAP:DSPE-mPEG in a molar ratio of 8 to 10:35 to 45:45 to 55:0.5 to 1.5, and more preferably, the lipid mixture may be comprised of HSPC:cholesterol:DOTAP:DSPE-mPEG in a molar ratio of 9:40:50:1, but is not limited thereto.

Within the above range, the optimal molar ratio or mol % may be appropriately selected depending on the type of protein encapsulated in the lipid nanoparticle. For example, when human iduronate-2-sulfatase (IDS) is encapsulated in the lipid nanoparticle, the lipid mixture may preferably be composed of HSPC:cholesterol:DOTAP:DSPE-mPEG in a molar ratio of 9:40:50:1.

In the present disclosure, the particle size of the lipid nanoparticle may affect the rate of drug release, biodistribution, mucosal adhesion, water uptake by cells, and buffer exchange and protein diffusion into the nanoparticles. For example, the LNP diameter may be in the range of 10 to 500 nm, preferably in the range of 70 to 120 nm, and more preferably in the range of 75 to 95 nm.

In the present disclosure, the polydispersity index (PDI) of the lipid nanoparticle is a measure of the heterogeneity of the molecular or particle size of the lipid mixture. For example, the PDI of the lipid nanoparticles may be in the range of 0.1 to 0.4, preferably in the range of 0.1 to 0.2, and more preferably in the range of 0.15 to 0.18.

In the present disclosure, the protein may be applied without any particular limitations, but may include, for example, an enzyme or a therapeutic protein. The therapeutic protein refers to a protein for treating or preventing a disease or disorder, and may be a recombinant protein, an antibody, a biosimilar, a biobetter, etc. In the present disclosure, the enzyme may be applied without any particular limitations, but may be any enzyme that is deficient or absent in the body, and in particular, the enzyme may be an enzyme for enzyme replacement therapy (ERT).

In the present disclosure, the enzyme may be applied without any particular limitations, but may be any enzyme that is deficient or absent in the body, and in particular, the enzyme may be an enzyme of enzyme replacement therapy (ERT).

As another aspect to achieve the objectives of the present disclosure, the present disclosure provides a lipid nanoparticle encapsulated with a protein, comprising a lipid mixture including HSPC; cholesterol; DOTAP; and DSPE-mPEG.

The “lipid nanoparticle” and “protein” are as described above.

The term “enzyme replacement therapy” as used herein refers to a treatment in which an enzyme that is deficient in the body is administered directly into the patient's vein. It is known as representative treatment for Fabry disease, Gaucher disease, Pompe disease, and Hunter syndrome, etc., which is called a lysosomal storage disorder (LSD), and is also used to treat rare congenital syndromes such as adenosine deaminase deficiency-induced severe combined immunodeficiency (ADA-SCID). In addition, enzyme replacement therapy may be utilized for enzyme or protein deficiencies in substrate reduction treatment, gene treatment, and bone marrow-derived stem cell transplantation, etc.

In the present disclosure, as an example, the enzyme may be a lysosomal enzyme that may be used in the treatment of a lysosomal storage disorder, and may be for example, but is not limited to, idursulfase, laronidase, elosulfase alpha, galsulfase, vestronidase alpha, agalsidase beta, agalsidase alpha, pegunigalsidase alpha, imiglucerase, taliglucerase alpha, alglucerase, velaglucerase, alglucosidase alpha, avalglucosidase alpha, sebelipase alpha, cerliponase alpha, or adenosine deaminase.

Specifically, the enzyme may be obtained by cloning the enzyme gene into a vector to produce an expression plasmid, transducing a host cell with the expression plasmid and culturing it, selecting and culturing a highly productive enzyme-expressing single clonal cell line, and then isolating and purifying the protein.

In the present disclosure, the enzyme may be for treating or preventing a lysosomal storage disorder. Lysosomes are intracellular organelles that are responsible for distributing the necessary components and excreting the unnecessary component among substances produced in the body. However, if there is a shortage of enzymes in the body, lysosomes are unable to perform this role properly, and in this case, impurities accumulate in the body, causing damage to body tissues and organs.

There are 50 or more of known lysosomal storage disorders. These disorders caused by genetic defects that inhibit the production of certain enzymes in lysosomes, resulting in an accumulation of products in the body, that eventually have serious effects on the skeleton, brain, skin, heart, and central nervous system, etc.

Specifically, the lysosomal storage disorders may be, but are not limited to, MPS I, MPS II, MPS IVA, MPS VI, MPS VII, Fabry disease, Gaucher disease, Gaucher disease type I, Pompe disease, lysosomal acid lipase deficiency (Wolman disease, CESD), neuronal ceroid lipofuscinosis (CLN2 disease), or severe combined immunodeficiency.

For example, Fabry disease is a progressive X-linked inborn error of glycosphingolipid metabolism caused by a deficiency of the lysosomal enzyme, α-galactosidase A (α-Gal A) due to a mutation in the α-Gal A gene (GLA). Despite being an X-linked disease, women may represent with varying degrees of clinical symptoms. Fabry disease is a rare disease with an estimated incidence of 1 in 40,000 in men and 1 in 117,000 in the whole population. Untreated patients with Fabry disease have a shortened life expectancy, with death usually occurring in the 40 or 50 years due to vascular disease affecting the kidneys, heart, and/or central nervous system. Enzyme deficiency causes intracellular accumulation of the substrate, globotriaosylceramide (GL-3) in vascular endothelium and visceral tissues throughout the body.

Heart disease caused by Fabry disease occurs in most men and many women. Early heart disease findings include left ventricular enlargement, valve involvement, and conduction abnormalities. Mitral regurgitation is typically the most common valve lesion that occurs in childhood or adolescence. Cerebrovascular symptoms are mainly due to multifocal small vessel involvement and may include thrombosis, transient ischemic attacks, basilar artery ischemia and aneurysms, seizures, hemiparesis, hemisensory loss, aphasia, labyrinthine dysfunction, or cerebral hemorrhage. The average age of onset of cerebrovascular symptoms is 33.8 years. Personality changes and psychotic behavior may occur with aging.

The current FDA-approved treatment for Fabry disease is enzyme replacement therapy (ERT), including agalsidase alfa (Replagal®; Shire Human Genetic Therapies), agalsidase beta (Fabrazyme®); Genzyme Corporation) and pegunigalcidase alpha (Elfabrio®); Protalix BioTherapeutics, Inc), etc. This type of ERT ensures that the patient's inadequate α-Gal A activity is supplemented by intravenous administration of a recombinant form of the enzyme. While the ERT is effective in many situations, it also has limitations. The ERT has not been proven to reduce the risk of stroke, the heart muscle responds slowly, and GL-3 clearance is limited in some cell types of the kidney. Some patients also develop an immune response to ERT.

As another example, Gaucher disease is also a representative lysosomal storage disorder, and is caused by the accumulation of glucocerebroside, which is a type of sphingolipid, mainly in the reticuloendothelial system, including the liver, spleen, central nervous system, skeleton, and lungs, due to a deficiency of β-glucocerebrosidase (β-GC), which is a hydrolytic enzyme of lysosomal enzyme. It is the most common lipid storage disorder.

Gaucher disease is a disease caused by mutations in the GBA gene and is inherited in an autosomal recessive manner, with clinical presentations of varying severity depending on the degree of deficiency in GBA enzyme activity. Gaucher disease has been divided into three clinical types according to the classification of Knudsen and Kaplan, depending on the presence or absence of central nervous system involvement, the rate of disease progression, and the age of onset, which have recently been classified into three groups: non-neuronopathic, acute or chronic neuronopathic. Here, the non-neuroinvasive type is called type 1, the acute neuroinvasive type is called type 2, and the chronic neuroinvasive type is called type 3.

Enzyme replacement therapy (ERT) is a typical treatment proven to be effective in treating Gaucher disease, and is known to be particularly helpful in improving hepatosplenomegaly and hematological findings in non-neuronopathic and chronic neuronopathic Gaucher disease. Gaucher disease is a disease that is the beginning of recombinant enzyme therapy. The acid β-glucosidase enzyme, imiglucerase (Cerezyme®, Genzyme Co.), has been commercialized since 1994 through recombinant DNA technology and has been used as a standard treatment for type I Gaucher disease, which is a non-neuronopathic form of the nervous system, until recently.

In Korea, Abcertin® (Isu Abxis, Seongnam, Korea), a biosimilar of imiglucerase, has been developed and commercialized, and VPRIV® (Takeda Pharmaceutical Company, Tokyo, Japan) is also used as a recombinant enzyme. These recombinant enzymes improve the symptoms of the reticuloendothelial system, resulting in improvement of hepatosplenomegaly. However, these enzymes do not pass through the blood-brain barrier, and therefore may not prevent the progression of neurological symptoms in patients with neuronopathic Gaucher disease. Additionally, it is known that the therapeutic effect is limited in patients with severe enlargement of lymph nodes in areas such as the abdomen or mediastinum, etc.

Other examples include mucopolysaccharidosis (MPS) I, II, IVA, VI, and VII, etc.

Mucopolysaccharidosis type I (MPS I) is a rare recessive genetic disease with an estimated incidence of 1 in 100,000 births (Moore D et al., 2008, Orphanet Journal of Rare Diseases 3). MPS I is caused by a deficiency of a-l-iduronidase (IDUA), an enzyme required for the liposomal catabolismof the very common complex polysaccharides heparan sulfate and dermatan sulfate. These polysaccharides, referred to as glycosaminoglycans (GAG), accumulate in the tissues of patients with MPS I, causing characteristic storage lesions and various disease sequelae. Patients may present with short stature, bone and joint deformities, coarse facial features, hepatosplenomegaly, cardiac valve disease, obstructive sleep apnea, recurrent upper respiratory tract infections, hearing impairment, carpal tunnel syndrome, and visual impairment due to corneal opacity (Beck M, et al., 2014, The natural history of MPS I: global perspectives from the MPS I Registry. Genetics in medicine: official journal of the American College of Medical Genetics 16(10):759-765). In addition, many patients develop symptoms associated with GAG storage in the central nervous system, which may include hydrocephalus, spinal cord compression, and, in some patients, cognitive impairment.

Mucopolysaccharidosis type II (Hunter syndrome/MPS II) is a rare X-linked recessive genetic disease that occurs in 0.5 to 1.3 per 100,000 male newborns. This progressive and devastating disease is caused by a genetic mutation in the IDS gene, which leads to a deficiency of the lysosomal storage enzyme, iduronate-2-sulfatase as an enzyme required for the lysosomal catabolismof heparan sulfate and dermatan sulfate.

These ubiquitous polysaccharides, called glycosaminoglycans (GAG), accumulate in the tissues and organs of patients with MPS II, causing characteristic storage lesions and various disease sequelae. Morbidity and mortality are high in this patient population. Death has been reported to occur at an average age of 11.7 years in patients with a severe phenotype (characterized by neurocognitive deterioration) and 21.7 years in patients with a mild or attenuated phenotype.

Mucopolysaccharidosis type IVA (MPS IVA; Morquio A syndrome) is an autosomal recessive lysosomal storage disorder caused by deficiency of N-acetylgalactosamine-6-sulfate sulfatase (GALNS) (Khan, etc., Mol Genet Metab., 2017; 120(1-2): 78-95). Deficiency of the enzymes results in a progressive accumulation of glycosaminoglycans (GAGs), chondroitin 6-sulfate (C6S), and keratan sulfate (KS), resulting in a unique skeletal dysplasia of the whole body with incomplete ossification and subsequent growth imbalance, which eventually results in a short neck and trunk, cervical spinal cord compression, tracheal obstruction, protruding scars, joint laxity, kyphoscoliosis, coxa valga, and genu valgum. Other clinical findings of the disease may include hearing loss, heart valve involvement, and corneal opacity. 200 or more of different mutations have been identified in patients, with a prevalence of about 1 in 250,000 in the United States.

By using the lipid nanoparticle according to the present disclosure in enzyme replacement therapy, treatment of the lysosomal storage disorder may be possible. Thus, from this respect, the present disclosure provides a method of treating a disease using an ERT enzyme. In addition, in addition to the lysosomal storage disorder, it is also possible to treat and prevent various diseases by effectively delivering proteins that are deficient or have low levels in the body. Thus, the lipid nanoparticle of the present disclosure, encapsulated with therapeutic proteins may be used to provide treatment methods for a variety of diseases. Such treatment methods may be achieved by intracellular delivery of the ERT enzyme or therapeutic protein, encapsulated into lipid nanoparticles.

As another aspect to achieve the objectives of the present disclosure, the present disclosure provides a composition for intracellular or in vivo delivery of a protein comprising a lipid nanoparticle encapsulated with the proteins or enzymes.

The “protein” and “lipid nanoparticle” are as described above.

In the present disclosure, the composition may further comprise a substance for increasing the efficiency of intracellular or in vivo delivery of the lipid nanoparticle encapsulated with proteins or enzymes, or may modify the form of the composition through additional improvement.

In another aspect to achieve the objectives of the present disclosure, the present disclosure provides a composition for preventing or treating lysosomal storage disorder, comprising the lipid nanoparticle encapsulated with the protein.

The “protein”, “lipid nanoparticle” and “lysosomal storage disorder” are as described above.

The term “prevention” as used herein refers to any act of inhibiting or delaying the onset of a lysosomal storage disorder by the administration of the pharmaceutical composition of the present disclosure. The term “treatment” as used herein refers to any act of ameliorating, alleviating, or beneficially altering a disease by the administration of the pharmaceutical composition of the present disclosure.

The term “pharmaceutical composition” as used herein refers to a composition prepared for the purpose of preventing or treating a disease, each of which may be formulated and used in various forms according to methods known in the art. For example, depending on the route of administration, it may be formulated into oral dosage forms, such as powders, granules, tablets, capsules, suspensions, emulsions, syrups, etc., and may be formulated and used in the form of an external preparation and a sterile injection solution. Specifically, the administration route may be any suitable route, including a topical route, an oral route, an intravenous route, an intramuscular route, and direct absorption through mucosal tissue, and may be used in combination of two or more routes. An example of a combination of two or more routes is a combination of two or more dosage forms of a drug according to the route of administration, for example, one drug is administered primarily by an intravenous route and the other is administered secondarily by a topical route.

The pharmaceutical compositions of the present disclosure may be formulated in the form of a pharmaceutical composition for the treatment or prevention of cancer, further comprising suitable a carrier, excipient or diluent conventionally used in the preparation of a pharmaceutical composition, wherein the carrier may comprise a non-naturally occurring carrier. Specifically, the pharmaceutical composition of the present disclosure may be formulated and used in the form of oral dosage forms such as powders, granules, tablets, capsules, suspensions, emulsions, syrups, aerosols, external preparations, suppositories, and sterile injectable solutions, respectively, according to conventional methods. A specific formulation of a pharmaceutical composition is known in the art and, for example, reference may be made to the literature [Remington's Pharmaceutical Sciences (19th ed., 1995)]. The foregoing literature is considered to be part of the present specification.

The pharmaceutical composition of the present disclosure may be administered to animals, such as rats, dogs, cats, cows, horses, pigs, and humans, and preferably humans, via various routes. Any mode of administration may be envisaged and includes, for example, but is not limited to, oral, intravenous, intraarterial, intramuscular or subcutaneous injection.

In the present disclosure, the treatment method also includes combinations with previously known therapies or co-administration with other treatment known in the art.

As another aspect to achieve the objectives of the present disclosure, the present disclosure provides a method for preparing a lipid nanoparticle encapsulated with a protein and a nanoparticle obtained by the method. Specifically, the method of preparing a lipid nanoparticle encapsulated with a protein comprises: (a) preparing a lipid mixture including HSPC; cholesterol; DOTAP; and DSPE-mPEG; (b) injecting a first solution in which the lipid mixture is dissolved in an alcohol solvent into an inlet of a microfluidic system; (c) injecting a second solution containing the protein into another inlet of the microfluidic system; and (d) purifying a lipid nanoparticle encapsulated with the protein.

The “protein” and “lipid nanoparticle” are as described above.

In the present disclosure, the alcohol solvent may include, but is not limited to, methanol, ethanol, propyl alcohol, or denatured alcohol, and may be preferably ethanol.

In the present disclosure, the flow rate ratio of an alcohol phase (a first solution) and an aqueous phase (a second solution) of the microfluidic system may be 1:3 to 1:5, but is not limited thereto, and may be preferably 1:3.

In the present disclosure, the flow rate of the microfluidic system may be 9 to 18 ml/min, but is not limited thereto, and may be preferably 15 ml/min.

In the present disclosure, the purifying of the lipid nanoparticle in the step (d) may comprise: removing the alcohol solvent in the step (b) and the encapsulated protein. Purification of the lipid nanoparticles may be performed using a tangential flow filtration system equipped with a column, and specifically, the purification may be performed by circulating the lipid nanoparticles through the column and adding fresh buffer (20 mM sodium phosphate, 137 mM sodium chloride, pH 6±0.2) at the same rate as the permeate exiting the column.

As another aspect to achieve the objectives of the present disclosure, the present disclosure provides a method for intracellular delivery of an ERT enzyme or a therapeutic protein using a lipid nanoparticle.

The “ERT”, “enzyme”, “therapeutic protein”, and “lipid nanoparticle” are as described above.

As another aspect to achieve the objectives of the present disclosure, the present disclosure provides a method for treating a disease using a lipid nanoparticle encapsulated with an ERT enzyme or a therapeutic protein.

The “ERT”, “enzyme”, “therapeutic protein”, and “lipid nanoparticle” are as described above.

In the present disclosure, the “disease” may be a lysosomal storage disorder. Additionally, in addition to the above lysosomal storage disorder, it is possible to treat and prevent various diseases by effectively delivering proteins that are deficient or have low levels in the body. Thus, the lipid nanoparticle of the present disclosure encapsulated with a therapeutic protein or an ERT enzyme may be used to provide a method of treating a variety of diseases.

The above treatment method may be achieved by intracellular delivery of an ERT enzymes or a therapeutic protein encapsulated in a lipid nanoparticle.

### Advantageous Effects

The present disclosure can be usefully used for the treatment and prevention of various diseases such as a lysosomal storage disorder by stably and effectively delivering various proteins such as therapeutic proteins or ERT enzymes into cells through lipid nanoparticles.

### DESCRIPTION OF DRAWINGS

FIG. 1 is an overall schematic diagram of lipid nanoparticle encapsulation according to a microfluidic system.

FIG. 2 shows the results of measuring the intracellular uptake of protein concentration after treating normal fibroblasts and fibroblasts from a patient with Hunter syndrome with IDS and IDS/LNPs of the present disclosure.

FIG. 3 shows the results of measuring the amount of intracellularly accumulated heparan sulfate after treating fibroblasts from a patient with Hunter syndrome with IDS and IDS/LNPs of the present disclosure.

### BEST MODE

Hereinafter, the present disclosure will be described in more detail with reference to the following Examples. However, these Examples are intended to illustrate the present disclosure by way of example, and the scope of the present disclosure is not limited to these Examples.

### Example 1. Preparation of Lipid Nanoparticles Encapsulated with Proteins

### 1.1. Production of Enzyme Protein

### 1.1.1. Development of IDS-Producing Cell Lines

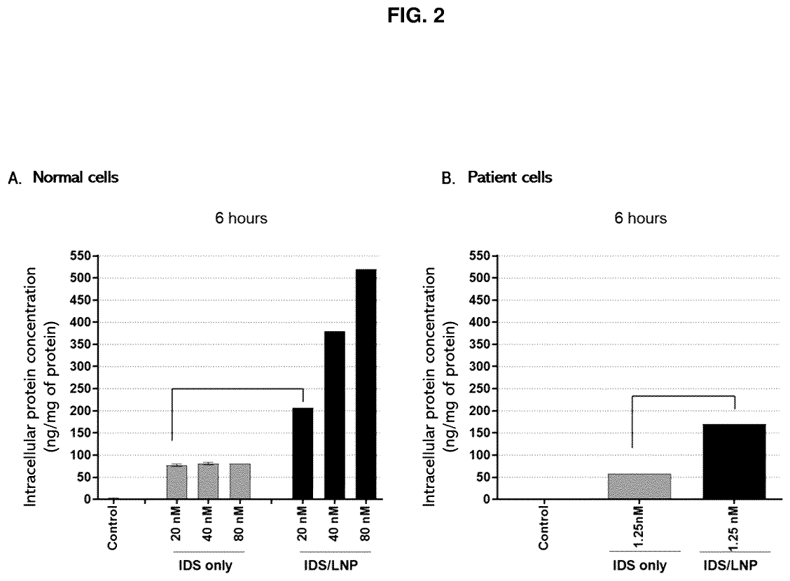

The human iduronate-2-sulfatase (hereinafter referred to as “IDS”) gene was synthesized and then cloned into Pangen's proprietary expression vector pPGXII (see FIG. 2 of Korean Patent No. 10-1385223) to construct an expression plasmid. Transduction of the CHO DG44 host cell with the expression plasmid was performed at a 24-well scale and conducted by using electroporation. After the cultivation in a 37° C., 5% CO₂ incubator, the cells were cultivated in selection medium to ensure that only the transformed cells may grow when they had grown sufficiently. After about two weeks, when the cells had grown sufficiently, a portion of the culture was taken and an IDS-expressing cell population was screened by ELISA assay.

Based on the results of the ELISA assay, the cell population was selected and subjected to single clonal selection to obtain a stable cell line with high expression efficiency. The cells were seeded into 96-well plates at 0.5 cells/well in culture medium, and the colonies formed after about 4 weeks were analyzed to select cell lines with high expression efficiency and cultured. When a sufficient number of cells were obtained, the expression efficiency of single clonal cell lines was compared and analyzed using ELISA assay, and the single clonal cell lines with high productivity were selected as candidate cell lines. The single clonal cell lines were confirmed to stably express proteins during long-term subculture of 90 days, and the cell line with the highest productivity among them was selected as the final cell line. The selected final cell line was dispensed into vials when a sufficient number of cells were secured to create a research cell bank, which was then stored in liquid nitrogen.

### 1.1.2. Cell Culture for IDS Production

The IDS-producing cell line was thawed and subcultured at 2 to 3 day intervals using subculture medium (EX-CELL (+8 mM L-glutamine)+1% 2× Feed A+). The subculture medium was used by adding 1% 2× Feed A+ adduct to EX-CELL medium supplemented with 8 mM L-glutamine.

After a total of 15 days of seed culture to obtain a sufficient number of cells, IDS was produced in 5 L culture volume in a 7.5 L bioreactor (Bioflo320, New Brunswick scientific). Cells were inoculated at a concentration of 1×10⁶ cells/mL during production, and the production medium was used by adding 12.5% 2× Feed A+ adduct to EX-CELL medium supplemented with 8 mM L-glutamine. The culture conditions were set at a culture temperature of 37° C., 30% DO (dissolved oxygen), pH 7.0 to 7.2, and a stirring speed of 150 rpm, and the culture was performed for a total of 11 days. During the culture period, cell count and viability were analyzed using a Vi-CELL™ counter. After pH and glucose and lactate contents were monitored by daily measurements, glucose content was maintained at 20 mM or more. After the culture was completed, the culture was harvested from the bioreactor and cells were removed using a C0HC depth filter and sterilized with a 0.22 μm PES filter.

### 1.1.3. Purification of IDS Protein

The cell culture was used to isolate and purify IDS proteins.

Chromatography was performed using a column packed with Capto MMC resin. The column was installed in an AKTA Pure (GE healthcare) and equilibrated with 20 mM sodium acetate/150 mM sodium chloride (pH 4.3) equilibration buffer, and then the culture adjusted to the same pH was loaded. The column was then washed and the IDS protein was eluted with 20 mM sodium acetate/150 mM sodium chloride (pH 5.1) elution buffer. Chromatography was performed using Blue Sepharose 6 FF as the eluent. The column was installed in an AKTA Pure (GE healthcare) and equilibrated with 20 mM sodium acetate/50 mM sodium chloride (pH 4.0) equilibration buffer, and then the samples diluted 1/3 with dilution buffer (20 mM sodium acetate (pH 4.0)) were loaded.

The column was then washed and the IDS protein was eluted with 20 mM sodium phosphate (pH 6.6) elution buffer. The eluted IDS protein was subjected to chromatography using Q Sepharose FF. The column was installed in an AKTA Pure (GE healthcare) and equilibrated with 20 mM sodium phosphate (pH 6.6) equilibration buffer, and then the samples were loaded. After washing the column with wash buffer (50 mM acetic acid (pH 3.0)), the IDS protein was obtained by flowing 50 mM acetic acid/150 mM sodium chloride (pH 3.0) elution buffer. Immediately before performing the next step of purification, the pH of the Q Sepharose FF eluate was reduced to 3.5 using 10% acetic acid, and the reaction was performed at room temperature for 180 minutes to perform the virus inactivation step. Immediately after the end of virus inactivation, the samples were diluted 1/3 with dilution buffer (50 mM sodium acetate/4,430 mM sodium chloride (pH 4.0)) and then subjected to Phenyl Sepharose chromatography. The column was installed in an AKTA Pure (GE healthcare) and equilibrated with 50 mM sodium acetate/3,000 mM sodium chloride (pH 4.0) equilibration buffer, and then the samples were loaded. The column was then washed and the IDS protein was eluted with 20 mM sodium phosphate (pH 6.0) elution buffer. The eluted IDS protein was prepared in 20 mM sodium phosphate/137 mM sodium chloride (pH 6.0) buffer at a concentration of about 2 mg/ml via the UF/DF process. After recovering the samples, Polysorbate 20 was added to an amount of 0.022%.

### 1.2. Preparation of Lipid Nanoparticles

Lipid nanoparticles (LNPs) were prepared using a microfluidic system by dissolving a cationic lipid mixture in ethanol at a specific concentration, mainly in the range of 2.5 to 7.5 mg/mL of total lipids.

Specifically, the selected lipid mixture was injected into one of the two inlets of a microfluidic system (a micromixer system using herringbone-shaped channels) manufactured in-house by MOOGENE MEDI, and cationic lipid nanoparticles were prepared to have a composition of 15% or less of hydrogenated soybean phosphatidiycholine (HSPC), 50% or less of cholesterol, 60% or less of dioleoyl-3-trimethylammonium propane (DOTAP), and 5% or less of DSPE-mPEG.

1.3. Preparation of Lipid Nanoparticles Encapsulated with Proteins

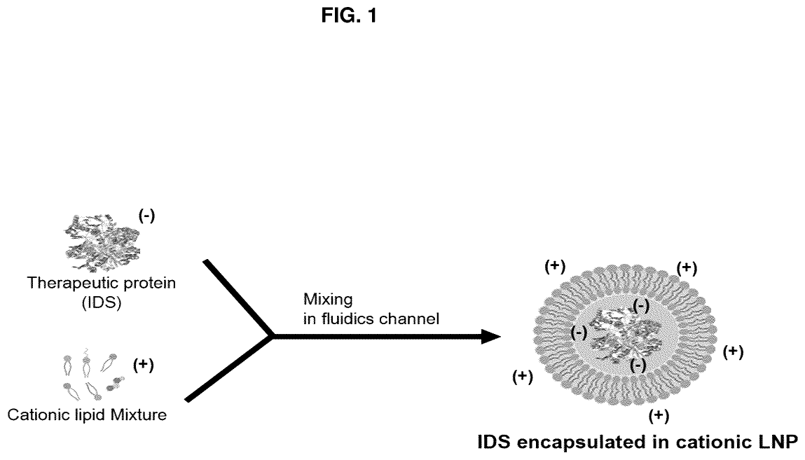

The encapsulation of the proteins via lipid nanoparticles was performed through a microfluidic system on a herringbone-shaped micromixer chip. An overall schematic diagram of lipid nanoparticle encapsulation according to flow rate in a microfluidic system is shown in FIG. 1.

The selected lipid mixture was dissolved in ethanol at a specific concentration, mainly in the range of 2.5 to 7.5 mg/ml of total lipid, and injected into one of the two inlets of the microfluidic system, and the aqueous phase (20 mM sodium phosphate, 137 mM sodium chloride, pH 6±0.2) wherein the protein was dissolved, was injected into a secondary inlet. Cationic lipid nanoparticles were prepared with a composition of 15% or less of HSPC, 50% or less cholesterol, 60% or less of DOTAP, and 5% or less of DSPE-mPEG. The size, polydispersity index (PDI), and encapsulation efficiency of the lipid nanoparticles prepared according to each lipid composition are as shown in Table 1 below.

TABLE 1

| | Lipid composition (mol %) | | | | | | |
|---|---|---|---|---|---|---|---|
| | | | | 18:0 PEG | Size | | Encapsulation |
| No. | HSPC | Cholesterol | DOTAP | 2000 PE | (nm) | PDI | Efficiency (%) |
| 1 | 8 | 40 | 50 | 2 | 102.9 | 0.2 | 67.4 |
| 2 | 8.5 | 40 | 50 | 1.5 | 113.1 | 0.11 | 602 |
| 3 | 9 | 40 | 50 | 1 | 93.3 | 0.17 | 65.9 |
| 4 | 9.5 | 40 | 50 | 0.5 | 110.5 | 0.16 | 76.9 |
| 5 | 10 | 40 | 50 | 0 | 94.2 | 0.1 | 22.7 |
| 6 | 18 | 30 | 50 | 2 | 85.8 | 0.40 | — |
| 7 | 28 | 20 | 50 | 2 | 80.8 | 0.88 | — |
| 8 | 38 | 10 | 50 | 2 | 177.9 | 0.54 | — |
| 9 | 48 | 0 | 50 | 2 | 132.6 | 0.57 | — |

*The ‘—’ in the encapsulation efficiency means not measured (NM).

Several production parameters were controlled via the microfluidic system software, including the flow rate ratio (the ratio between the alcoholic and the aqueous phases) and the total flow rate (the rate at which the two inlets are injected through the chip). Specifically, the flow rate ratio of alcohol phase:aqueous phase=1:3 to 1:5 and the flow rate of 9 to 18 ml/min were tested to select the optimal production parameters (Table 2 and Table 3).

TABLE 2

| | Flow rate | Size | | Encapsulation |
|---|---|---|---|---|
| No. | ratio | (nm) | PDI | Efficiency (%) |
| 1 | 1:3 | 98.7 | 0.22 | 76.7 |
| 2 | 1:4 | 108.5 | 0.12 | 70.0 |
| 3 | 1:5 | 109.5 | 0.17 | 62.8 |

TABLE 3

| | Flwo rate | Size | | Encapsulation |
|---|---|---|---|---|
| No. | (ml/min) | (nm) | PDI | Efficiency (%) |
| 1 | 9 | 82.4 | 0.40 | 63.5 |
| 2 | 12 | 69.6 | 0.29 | 69.9 |
| 3 | 15 | 97.1 | 0.22 | 68.4 |
| 4 | 18 | 77.1 | 0.34 | 75.1 |

### 1.4. Purification of Lipid Nanoparticles

The prepared lipid nanoparticles were purified using a tangential flow filtration system (KrosFlo® KR2i TFF System by Repligen) equipped with a modified polyethersulfone (mPES) hollow fiber column with a pore size of 750 kD. To remove ethanol and proteins not encapsulated in the lipid nanoparticles, lipid nanoparticle samples were circulated through the column and samples were purified by adding fresh buffer (20 mM sodium phosphate, 137 mM sodium chloride, pH 6±0.2) at the same rate as the permeate exiting the column.

### Example 2. Intracellular Uptake of IDS-LNP

[Iduronate-2-sulfatase protein-cationic lipid nanoparticles] (hereinafter, ‘IDS/LNPs’) where IDS, an enzyme protein deficient in a patient with mucopolysaccharidosis type II (Hunter syndrome/MPS II) was encapsulated in cationic lipid nanoparticles, were prepared by the method of Example 1.

Fibroblasts form normal human (CCD-986Sk, Korean Cell line Bank, KCLB21947) and fibroblasts from a patent with Hunter syndrome (GM00615, Coriell Institute, hereinafter referred to as “patient cells”) were treated with IDS and IDS/LNPs at various concentrations ranging from 1.25 to 80 nM for 6 hours, and the amount of intracellular uptake of protein was quantified using ELISA method.

Specifically, the cells used in the experiments were cultured at 5% CO₂, 37° C. in Iscove's Modified Dulbecco's Medium (IMDM) medium supplemented with 10% fetal bovine serum (FBS) and 1% penicillin-streptomycin antibiotics for fibroblasts from normal humans and in Minimum Essential Medium (MEM) with Earle's salts, L-glutamine, sodium bicarbonate supplemented with 10% fetal bovine serum (FBS) for cells from a patient with Hunter syndrome, respectively. Cells inoculated in 60 mm² cell culture plates (2.5×10⁵ cells/plate) 24 hours before IDS/LNPs treatment were used, and on the day of drug treatment, IDS control drug (ELAPRASE), IDS, and IDS/LNPs were added to 5% FBS-IMDM or MEM culture medium and cultured at 37° C. for 24 hours. Passage number of the patient cells used in the experiment was less than 10.

After 24 hours of reaction, the culture was removed, washed once or twice with PBS, and then treated with 0.25% Trypsin-EDTA, centrifuged, and washed with PBS to collect only the cells. The collected cells were lysed in RIPA buffer containing protease inhibitors and phosphatase inhibitors (Sigma), centrifuged, and only the supernatant was collected and used as samples for analysis. In the samples for analysis (whole cell lysates), intracellular uptake of IDS and BCA (Bicinchoninic acid) was quantified using a Human Iduronate 2-Sulfatase/IDS ELISA kit (R&D systems, Cat. #DY2449-05) and the amount of protein was quantified using a BCA (Bicinchoninic acid) protein assay kit (Thermo Scientific), respectively. The result was expressed as ng of intracellular uptake of IDS protein per mg of protein.

As a result, it was confirmed that IDS/LNPs treatment resulted in significantly higher amounts of intracellular uptake of IDS protein in normal human and patient, compared to the case of IDS treatment alone (FIG. 2).

### Example 3. Confirmation of Cellular Activity of IDS/LNPs

In patients with mucopolysaccharidosis type II (Hunter syndrome/MPS II), the deficiency of IDS results in the accumulation of polysaccharides called intracellular glycosaminoglycan (GAG), and heparan sulfate is a type of representative GAG.

To measure the cellular activity of IDS/LNPs, patient cells were treated with IDS protein and IDS/LNPs at concentrations of 5 to 40 nM for 24 hours, and the decreased amount of intracellular accumulated heparan sulfate was quantified by ELISA method.

Specifically, patient cells used in the experiments were cultured in Minimum Essential Medium (MEM) with Earle's salts, L-glutamine, sodium bicarbonate supplemented with 10% fetal bovine serum (FBS) at 37° C. under 5% CO₂. Cells inoculated in 60 mm² cell culture plates (2.5×10⁵ cells/plate) 24 hours before IDS/LNPs treatment were used, and on the day of drug treatment, IDS control drug (ELAPRASE), IDS, and IDS/LNPs were added to 5% FBS-MEM culture medium and cultured at 37° C. for 24 hours. Passage number of the patient cells used in the experiment was less than 10.

After 24 hours of reaction, the culture was removed, washed once or twice with PBS, and then treated with 0.25% Trypsin-EDTA, centrifuged, and washed with PBS to collect only the cells. The collected cells were lysed in RIPA buffer containing protease inhibitors and phosphatase inhibitors (Sigma), centrifuged, and only the supernatant was collected and used as samples for analysis. In the samples for analysis (whole cell lysates), intracellular heparan sulfate was quantified using a Human HS (Heparan Sulfate) ELISA kit (MyBioSource, Cat. #MBS2515971), and the amount of protein was quantified using a BCA protein assay kit (Thermo Scientific), respectively. The result was expressed as HS content (ng/mg) corrected by dividing HS concentration (ng/ml) by protein concentration (mg/ml).

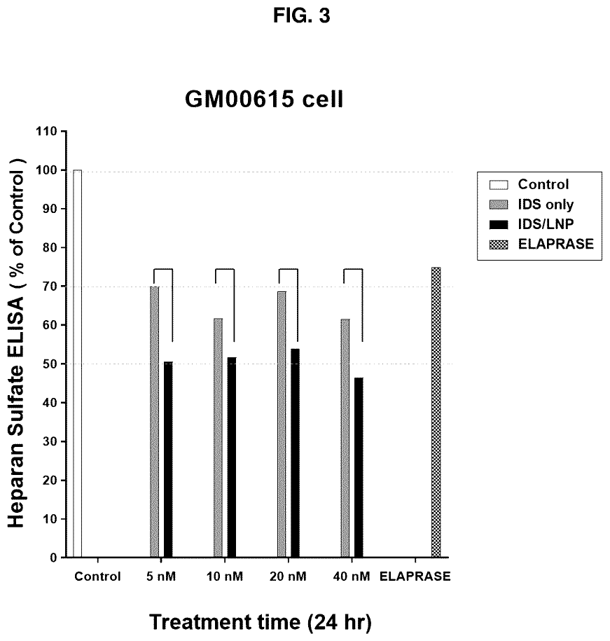

As a result, it was confirmed that the activity of the IDS/LNPs treatment group was significantly higher than that of the IDS protein treatment group (FIG. 3).

As described above, as the specific parts of the present disclosure have been described in detail, it will be obvious to those skilled in the art that these specific descriptions are merely preferred embodiments and the scope of the present disclosure is not limited thereby. Therefore, it will be said that the substantial scope of the present disclosure is defined by the appended claims and their equivalents.

1. A lipid nanoparticle for intracellular or in vivo delivery of a protein, comprising a lipid mixture including hydrogenated soybean phosphatidylcholine (HSPC); cholesterol; dioleoyl-3-trimethylammonium propane (DOTAP); and 1,2-distearoyl-sn-glycero-3-phosphoethanolamine-N-methoxy(polyethylene glycol) (DSPE-mPEG).

2. The lipid nanoparticle of claim 1, wherein the protein is an enzyme or a therapeutic protein.

3. The lipid nanoparticle of claim 1, wherein the HSPC is included in an amount of 8 to 15 mol % of the lipid mixture.

4. The lipid nanoparticle of claim 1, wherein the cholesterol is included in an amount of 30 to 50 mol % of the lipid mixture.

5. The lipid nanoparticle of claim 1, wherein the lipid mixture is composed of HSPC:cholesterol:DOTAP:DSPE-mPEG in a molar ratio of 8 to 15:30 to 50:40 to 60:0.5 to 5.

6. A lipid nanoparticle encapsulated with a protein, comprising a lipid mixture including HSPC; cholesterol; DOTAP; and DSPE-mPEG.

7. The lipid nanoparticle of claim 6, wherein the protein is an enzyme or a therapeutic protein.

8. The lipid nanoparticle of claim 7, wherein the enzyme is an enzyme for enzyme replacement therapy (ERT).

9. The lipid nanoparticle of claim 8, wherein the enzyme is a lysosomal enzyme.

10. The lipid nanoparticle of claim 9, wherein the enzyme is idursulfase, laronidase, elosulfase alpha, galsulfase, vestronidase alpha, agalsidase beta, agalsidase alpha, pegunigalsidase alpha, imiglucerase, taliglucerase alpha, alglucerase, velaglucerase, alglucosidase alpha, avalglucosidase alpha, sebelipase alpha, cerliponase alpha, or adenosine deaminase.

11. The lipid nanoparticle of claim 6, wherein the lipid mixture is comprised of HSPC:cholesterol:DOTAP:DSPE-mPEG in a molar ratio of 8 to 15:30 to 50:40 to 60:0.5 to 5.

12. The lipid nanoparticle of claim 6, wherein the lipid nanoparticle is for treating a lysosomal storage disorder.

13. The lipid nanoparticle of claim 12, wherein the lysosomal storage disorder is MPS I, MPS II, MPS IVA, MPS VI, MPS VII, Fabry disease, Gaucher disease, Gaucher disease type I, Pompe disease, lysosomal acid lipase deficiency (Wolman disease, CESD), neuronal ceroid lipofuscinosis (CLN2 disease), or severe combined immunodeficiency.

14. A composition for intracellular or in vivo delivery of a protein, comprising the lipid nanoparticle of claim 6.

15. A pharmaceutical composition for preventing or treating a lysosomal storage disorder, comprising the lipid nanoparticle of claim 6.

16. (canceled)

17. A method for preparing a lipid nanoparticle encapsulated with a protein, comprising:
(a) preparing a lipid mixture including HSPC; cholesterol; DOTAP; and DSPE-mPEG;
(b) injecting a first solution in which the lipid mixture is dissolved in an alcohol solvent into an inlet of a microfluidic system;
(c) injecting a second solution containing the protein into another inlet of the microfluidic system; and
(d) purifying a lipid nanoparticle encapsulated with the protein.

18. The method of claim 17, wherein the lipid mixture in the step (a) is composed of HSPC:cholesterol:DOTAP:DSPE-mPEG in a molar ratio of 8 to 15:30 to 50:40 to 60:0.5 to 5.

19. The method of claim 17, wherein a flow rate ratio of an alcoholic phase and an aqueous phase of the microfluidic system is 1:3 to 1:5.

20. The method of claim 17, wherein a flow rate of the microfluidic system is 9 to 18 ml/min.

21. The method of claim 17, wherein the purifying of the lipid nanoparticle in the step (d) comprises removing the alcohol solvent in the step (b) and a protein not encapsulated in the lipid nanoparticle.

22-23. (canceled)
